SEQUENCE LISTING (9 sequences)

SEQ ID NO: 1 - DNA, 1992 bp, Homo sapiens
atggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacagccctccagccaagaagaaatccacaggttcctccacatggcccctggaccctggggtagaggtgaccctgacgatgaaagcggccagtggtagcacaggcgaccagaaggttcagatttcatactacggacccaagactccaccagtcaaagctctactctacctcaccgcggtggaaatctccctgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgagtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctga

SEQ ID NO: 2 - DNA, 23 bp, Artificial Sequence
  Synthetic
gggctggcaagccacgtttggtg

SEQ ID NO: 3 - DNA, 37 bp, Artificial Sequence
  Synthetic
ttggtaccgaattcgcggccgcgagctcttgcttgcc

SEQ ID NO: 4 - DNA, 36 bp, Artificial Sequence
  Synthetic
gactctccaaggaacnnsggcctgaaggagtttccc

SEQ ID NO: 5 - DNA, 37 bp, Artificial Sequence
  Synthetic
aaactccttcaggccsnngttccttggagagtcgaag

SEQ ID NO: 6 - DNA, 32 bp, Artificial Sequence
  Synthetic
cttcttcacctaccacatcnnscatggggagg

SEQ ID NO: 7 - DNA, 29 bp, Artificial Sequence
  Synthetic
ccccatgsnngatgtggtaggtgaagaag

SEQ ID NO: 8 - DNA, 36 bp, Artificial Sequence
  Synthetic
cttcacctaccacatcnnsnnsggggaggtgcactg

SEQ ID NO: 9 - DNA, 36 bp, Artificial Sequence
  Synthetic
cagtgcacctccccsnnsnngatgtggtaggtgaag

(19) United States
(12) Patent Application Publication
(10) Pub. No.: US 2009/0238813 A1
(43) Pub. Date: Sep. 24, 2009

(54) Compositions And Methods For Engineered Human Arginine Deiminases

(75) Inventors: George Georgiou, Austin, TX (US); Everett Stone, Austin, TX (US); Walter Fast, Austin, TX (US)

Correspondence Address: Peter G. Carroll;MEDLEN & CARROLL, LLP, Suite 350, 101 Howard Street, San Francisco, CA, 94105, US

(73) Assignee: Board of Regents, The University of Texas System

(21) Appl. No.: 12/327,063

(22) Filed: Dec. 3, 2008

Related U.S. Application Data
(60) Provisional application No. 61/009,374, filed on Dec. 28, 2007.

(51) Int. Cl. A61K 38/46; C12N 9/14; C12P 19/34; C40B 30/06

(52) U.S. Cl. 424/94.6; 435/195; 435/91.5; 506/10

(57) ABSTRACT

The present invention discloses the engineering of a human enzyme with arginine hydrolytic activity suitable for human therapy. An enzyme comprising of a human sequence is not likely to induce adverse immunological responses and thus is expected to constitute a superior therapeutic. Since the human genome does not encode arginases with the proper high affinity catalytic properties (i.e., for example, a low Km and high catalytic activity, kcat) an appropriate arginase can be engineered by modifying an enzyme with related catalytic activity. For example, the human enzyme PAD4 can hydrolyze arginine in peptide substrates but does not have activity for free arginine. First, a high throughput assay was developed for detecting arginine activity by monitoring the formation of the hydrolytic product citrulline. Then, using a combination of rational design and iterative mutation and screening PAD4 mutants were identified and isolated exhibiting high affinity free arginine metabolic activity. These mutants did not retain activity for their original substrate, peptidyl arginine.

### FIELD OF THE INVENTION

This invention is related to compositions and methods for the treatment of cancer. In some embodiments, the invention contemplates human arginine degrading enzyme variants. For example, a rationally guided and directed evolution approach may be employed to create a human peptidyl arginine deiminase IV (PAD4) with arginine deiminase (ADI) activity.

### BACKGROUND

Melanomas, hepatocellular carcinomas (HCCs) and renal cell carcinomas (RCCs) are among the deadliest forms of cancer and are highly resistant to current chemotherapies, making new drugs to treat these types of cancer of significant interest. However, these carcinomas have been shown to be auxotrophic for arginine due to loci involved in argininosuccinate synthetase expression.

Thus, systemic arginine depletion is an attractive chemotherapeutic strategy targeting malignant auxotrophic cells without the use of toxins. A bacterial enzyme, arginine deiminase (ADI), which catalyses the hydrolysis of arginine to citrulline and ammonia has been employed for eliminating arginine in serum systemically. Phase I/II studies with the bacterial ADI enzyme have been completed successfully. However, the bacterial enzyme is immunogenic in humans and can result in allergic reactions and the production of neutralizing antibodies.

What is needed in the art is a human enzyme capable of degrading free arginine in blood and thus is effective as a chemotherapeutic agent.

### SUMMARY

This invention is related to compositions and methods for the treatment of cancer. In some embodiments, the invention contemplates human arginine degrading enzyme variants. For example, a rationally guided and directed evolution approach may be employed to create a human peptidyl arginine deiminase IV (PAD4) with arginine deiminase activity.

In one embodiment, the present invention contemplates a composition comprising a mutated human peptidyl arginine deiminase IV enzyme, wherein said enzyme comprises a high affinity free arginine binding site. In one embodiment, the mutated enzyme comprises at least two altered amino acid residues when compared to a wild type human peptidyl arginine deiminase IV enzyme. In one embodiment, the mutated enzyme comprises catalytic activity in the hydrolysis of arginine. In one embodiment, the altered amino acid residue comprises an altered AA³⁷⁴. In one embodiment, the altered AA³⁷⁴ is selected from the group consisting of arginine, lysine, serine, or proline. In one embodiment, the altered amino acid comprises an altered AA⁶³⁹. In one embodiment, the altered AA⁶³⁹ is selected from the group consisting of arginine, asparagine, lysine, serine, glutamic acid, histidine, methionine, valine, isoleucine, or tyrosine. In one embodiment, the altered amino acid comprises an altered AA⁶⁴⁰. In one embodiment, the altered AA⁶⁴⁰ is selected from the group consisting of glycine, asparagine, valine, lysine, arginine, or histidine.

In one embodiment, the present invention contemplates a composition comprising a human peptidyl arginine deiminase IV enzyme comprising at least two mutations, wherein said mutations are at amino acid positions selected from the group consisting of Arg³⁷⁴, Arg⁶³⁹, and His⁶⁴⁰. In one embodiment, the enzyme further comprises a high affinity free arginine binding site. In one embodiment, the enzyme comprises arginine deiminase activity. In one embodiment, the Arg³⁷⁴ mutation creates a first altered amino acid selected from the group consisting of lysine, serine, and proline. In one embodiment, the Arg⁶³⁹ mutation creates a second altered amino acid selected from the group consisting of asparagine, lysine, serine, glutamic acid, histidine, methionine, valine, isoleucine, and tyrosine. In one embodiment, the His⁶⁴⁰ mutation creates a third altered amino acid selected from the group consisting of glycine, asparagine, valine, lysine, and arginine.

In one embodiment, the present invention contemplates a method, comprising: a) providing a wild type nucleic acid sequence encoding a wild type human amino acid sequence, wherein said wild type amino acid sequence comprises a high catalytic activity for peptidyl arginine; and b) mutagenizing the wild type nucleic acid sequence to create a mutated nucleic acid sequence, wherein said mutated nucleic acid sequence encodes a mutated human amino acid sequence, wherein said mutated amino acid sequence comprises high catalytic activity for L-Arg. In one embodiment, the mutated human amino acid sequence comprises at least 95% of said wild type human amino acid sequence. In one embodiment, the wild type human amino acid sequence comprises an peptidyl arginine deiminase IV enzyme. In one embodiment, The mutated human amino acid sequence comprises a k_{cat} of 4-6 s⁻¹ for free arginine. In one embodiment, the mutated human amino acid sequence comprises at least two altered amino acid residues.

In one embodiment, the present invention contemplates a method: a) providing; i) a wild type nucleic acid sequence encoding a wild type human amino acid sequence, wherein said wild type amino acid sequence comprises a high affinity binding site for a first substrate; ii) a directed evolution technique capable of mutagenizing the wild type nucleic acid sequence; and b) mutagenizing the wild type nucleic acid sequence to create a mutated nucleic acid sequence, wherein said mutated nucleic acid sequence encodes a mutated human amino acid sequence, wherein said mutated amino acid sequence comprises a high affinity binding site for a second substrate. In one embodiment, the mutated human amino acid sequence comprises at least 95% of the wild type human amino acid sequence. In one embodiment, the wild type human amino acid sequence comprises an peptidyl arginine deiminase IV enzyme. In one embodiment, the mutated human amino acid sequence confers a k_{cat} of 4 s⁻¹ for free arginine. In one embodiment, the mutated human amino acid sequence comprises at least two altered amino acid residues. In one embodiment, the altered amino acid residue comprises AA³⁷⁴. In one embodiment, the AA³⁷⁴ is selected from the group consisting of arginine, lysine, serine, or proline. In one embodiment, the altered amino acid comprises AA⁶³⁹. In one embodiment, the AA⁶³⁹ is selected from the group consisting of arginine, asparagine, lysine, serine, glutamic acid, histidine, methionine, valine, isoleucine, or tyrosine. In one embodiment, the altered amino acid comprises AA⁶⁴⁰. In one embodiment, the AA⁶⁴⁰ is selected from the group consisting of glycine, asparagine, valine, lysine, arginine, or histidine. In one embodiment, the directed evolution comprises iterative rounds of structure guided mutagenesis. In one embodiment, the structure guided mutagenesis further comprises screening to isolate a clone that expresses an enzyme having the highest catalytic activity. In one embodiment, the screening identifies a clone having an optimized catalytic activity (i.e., for example, highest activity and/or desired activity). In one embodiment, the directed evolution comprises random mutagenesis. In one embodiment, the random mutagenesis comprises error-prone polymerase chain reaction. In one embodiment, the random mutagenesis comprises amino acid randomization. In one embodiment, the directed evolution comprises gene shuffling. In one embodiment, the method further comprises a high throughput arginine deiminase activity assay.

In one embodiment, the present invention contemplates a method, comprising: a) providing: i) a library of bacterial cells transfected by oligonucleotides encoding a mutated human peptidyl arginine deiminase IV enzyme; and iii) an assay capable of detecting free arginine deiminase activity; b) expressing said oligonucleotides from said bacterial cells, thereby producing the mutated enzymes; and c) using the assay to identify the bacterial cells expressing mutated enzymes capable of metabolizing free arginine. In one embodiment, the bacterial cells are transfected using a pGEX-6p1 vector. In one embodiment, the bacterial cell comprise E. coli cells. In one embodiment, the oligonucleotides were constructed by overlap extension polymerase chain reaction. In one embodiment, the oligonucleotides comprise randomized codons encoding an amino acid residue selected from the group consisting of position 374 and position 639. In one embodiment, the E. coli cells comprise DH5α E. coli cells. In one embodiment, the randomized codon encoding amino acid position 374 is selected from the group consisting of AAG, AGC, CCG, TCC, and ATG. In one embodiment, the randomized codon encoding amino acid position 639 is selected from the group consisting of TTG, AAC, TCC, CAC, GAG, and AAC.

In one embodiment, the present invention contemplates a method, comprising: a) providing; i) a human patient comprising a population of cancer cells, wherein said cancer cells are deficient in the synthesis of arginine; ii) a mutated human peptidyl arginine deiminase IV enzyme, wherein said enzyme is capable of degrading free arginine; and b) administering said enzyme to said patient under conditions that said population of cancer cells is reduced. In one embodiment, the administering further created the arginine deficiency. In one embodiment, the enzyme is mutated in at least two amino acid residues. In one embodiment, the mutated amino acid residues are selected from the group consisting of from AA³⁷⁴, AA⁶³⁹, and AA⁶⁴⁰. In one embodiment, the AA³⁷⁴ is selected from the group consisting of arginine, lysine, serine, or proline. In one embodiment, the AA⁶³⁹ is selected from the group consisting of arginine, asparagine, lysine, serine, glutamic acid, histidine, methionine, valine, isoleucine, or tyrosine. In one embodiment, the AA⁶⁴⁰ is selected from the group consisting of glycine, asparagine, valine, lysine, arginine, or histidine. In one embodiment, the administering comprises a pharmaceutical composition. In one embodiment, the population of cancer cells comprise hepatic carcinoma cancer cells. In one embodiment, the population of cancer cells comprise renal carcinoma cancer cells.

### DEFINITIONS

The term “instructions for using said kit for said detecting the presence or absence of a variant arginase nucleic acid or polypeptide in said biological sample” as used herein, includes instructions for using the reagents contained in the kit for the detection of variant and wild type arginase polypeptides. In some embodiments, the instructions further comprise the statement of intended use required by the U.S. Food and Drug Administration (FDA) in labeling in vitro diagnostic products.

The term “gene” as used herein, refers to a nucleic acid (e.g., DNA) sequence that comprises coding sequences necessary for the production of a polypeptide or, RNA (e.g., including but not limited to, mRNA, tRNA and rRNA). The polypeptide or RNA can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “PAD4 gene” as used herein, refers to a full-length PAD4 nucleotide sequence encoding the PAD4 wild type amino acid sequence (e.g., contained in SEQ ID NO: 1). Furthermore, the terms “PAD4 nucleotide sequence” or “PAD4 polynucleotide sequence” encompasses DNA, cDNA, and RNA (e.g., mRNA) sequences. A PAD4 polynucleotide sequence may further be defined as containing naturally occurring polymorphisms (i.e., for example, human PAD4 polymorphisms).

The term “polymorphism” as used herein, refers to any gene containing a coding region with one (i.e., for example, a single nucleotide polymorphism or SNP) or more different nucleotide sequences (i.e., for example, resulting in different alleles) when compared to the wild type nucleotide sequence. Such different nucleotide sequences may be expressed to produce proteins that may have the same or different functional activity. For example, some nucleotides containing a polymorphism may express a protein having an increased activity, while other expressed protein may have a decreased activity.

The term “amino acid sequence” as used herein, refers to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The term “wild-type” as used herein, refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The terms “nucleic acid molecule encoding,” “DNA sequence encoding,” and “DNA encoding” as used herein, refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′end” if its 5′ phosphate is not linked to the 3′ oxygen of a mononucleotide pentose ring and as the “3′end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The terms “an oligonucleotide having a nucleotide sequence encoding a gene” and “polynucleotide having a nucleotide sequence encoding a gene,” as used herein, mean a nucleic acid sequence comprising the coding region of a gene or, in other words, the nucleic acid sequence that encodes a gene product. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The term “regulatory element” as used herein, refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

The terms “complementary” or “complementarity” as used herein, when in reference to polynucleotides (i.e., a sequence of nucleotides) related by the base-pairing rules. For example, for the sequence 5′-“A-G-T-3′,” is complementary to the sequence 3′-“T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” as used herein, refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (i.e., for example, Southern or Northern blot, solution hybridization and the like) under conditions of low stringency. A substantially homologous sequence or probe will compete for and inhibit the binding (i.e., the hybridization) of a sequence completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target. Numerous equivalent conditions may be employed to comprise “low stringency” conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

The term “substantially homologous” as used herein, refers to any probe that can hybridize to either or both strands of the double-stranded nucleic acid sequence or can hybridize to a single stranded nucleic acid sequence under conditions of low stringency.

The term “competes for binding” as used herein, is used in reference to a first polypeptide with an activity which binds to the same substrate as does a second polypeptide with an activity, where the second polypeptide is a variant of the first polypeptide or a related or dissimilar polypeptide. The efficiency (e.g., kinetics or thermodynamics) of binding by the first polypeptide may be the same as or greater than or less than the efficiency substrate binding by the second polypeptide. For example, the equilibrium binding constant (K_{D}) for binding to the substrate may be different for the two polypeptides. The term “Kₘ” as used herein refers to the Michaelis-Menton constant for an enzyme and is defined as the concentration of the specific substrate at which a given enzyme yields one-half its maximum velocity in an enzyme catalyzed reaction.

The term “hybridization” as used herein, is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

The term “Tₘ” as used herein, is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

The term “stringency” as used herein, is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

The term “high stringency conditions” as used herein, when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

The term “medium stringency conditions” as used herein, when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

The term “low stringency conditions” as used herein, comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed. The present invention is not limited to the hybridization of probes of about 500 nucleotides in length. The present invention contemplates the use of probes between approximately 10 nucleotides up to several thousand (e.g., at least 5000) nucleotides in length.

The term “reference sequence” as used herein, refers to any defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by one of many homology algorithms. Smith et al., Adv. Appl. Math. 2: 482 (1981); Needleman et al., J. Mol. Biol. 48:443 (1970); Pearson et al., Proc. Natl. Acad. Sci. (U.S.A.) 85:2444 (1988), including computerized implementations of these algorithms (i.e., for example, GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” as used herein, means that two polynucleotide sequences are identical (i.e., on a nucleotide-by-nucleotide basis) over the window of comparison.

The term “percentage of sequence identity” as used herein, is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “substantial identity” as used herein, denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention (e.g., PAD4).

The term “substantial identity” as used herein, when applied to polypeptides, means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “fragment” as used herein, refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, and span the portion of the polypeptide required for intermolecular binding of the compositions (claimed in the present invention) with its various ligands and/or substrates.

The term “polymorphic locus” as used herein, is a locus present in a population that shows variation between members of the population (i.e., the most common allele has a frequency of less than 0.95). In contrast, a “monomorphic locus” is a genetic locus at little or no variations seen between members of the population (generally taken to be a locus at which the most common allele exceeds a frequency of 0.95 in the gene pool of the population).

The term “genetic variation information” or “genetic variant information” as used herein, refers to the presence or absence of one or more variant nucleic acid sequences (e.g., polymorphism or mutations) in a given allele of a particular gene (e.g., the PAD4 gene).

The term “detection assay” as used herein, refers to any assay for detecting the presence of absence of variant nucleic acid sequences (e.g., polymorphism or mutations) in a given allele of a particular gene (e.g., the PAD4 gene). Examples of suitable detection assays include, but are not limited to, those described below.

The term “naturally-occurring” as used herein, as applied to an object, refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “amplification” as used herein, refers to a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out. Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase. D. L. Kacian et al., Proc. Natl. Acad. Sci. USA 69:3038 (1972). Other nucleic acid will not be replicated by this amplification enzyme. Similarly, in the case of T7 RNA polymerase, this amplification enzyme has a stringent specificity for its own promoters. Chamberlin et al., Nature 228:227 (1970]. In the case of T4 DNA ligase, the enzyme will not ligate the two oligonucleotides or polynucleotides, where there is a mismatch between the oligonucleotide or polynucleotide substrate and the template at the ligation junction. D. Y. Wu and R. B. Wallace, Genomics 4:560 (1989). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences. H. A. Erlich (ed.), PCR Technology, Stockton Press (1989).

The term “amplifiable nucleic acid” as used herein, is used in reference to nucleic acids that may be amplified by any amplification method. It is contemplated that “amplifiable nucleic acid” will usually comprise “sample template.”

The term “sample template” as used herein, refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

The term “primer” as used herein, refers to any oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” as used herein, refers to any oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein, refers to any nucleic acid sequence or structure to be detected or characterized. Thus, the “target” is sought to be sorted out from other nucleic acid sequences. A “segment” is defined as a region of nucleic acid within the target sequence.

The term “polymerase chain reaction” (“PCR”) as used herein, refers to methods of nucleic acid amplification. K. B. Mullis U.S. Pat. Nos. 4,683,195, 4,683,202, and 4,965,188, hereby incorporated by reference. These methods increase the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” as used herein, refer to any resultant mixture of compounds after two or more cycles of the PCR steps of denaturation, annealing and extension are complete. These terms encompass the case where there has been amplification of one or more segments of one or more target sequences.

The term “amplification reagents” as used herein, refer to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The terms “restriction endonucleases” and “restriction enzymes” as used herein, refer to bacterial enzymes, each of which cut double-stranded DNA at or near a specific nucleotide sequence.

The term “recombinant DNA molecule” as used herein, refers to a DNA molecule that is comprised of segments of DNA joined together by means of molecular biological techniques.

The term “antisense” as used herein, is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” as used herein in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding PAD4 includes, by way of example, such nucleic acid in cells ordinarily expressing PAD4 where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

The term “portion of a chromosome” as used herein, refers to any discrete section of the chromosome. Chromosomes are divided into sites or sections by cytogeneticists as follows: the short (relative to the centromere) arm of a chromosome is termed the “p” arm; the long arm is termed the “q” arm. Each arm is then divided into 2 regions termed region 1 and region 2 (region 1 is closest to the centromere). Each region is further divided into bands. The bands may be further divided into sub-bands. A portion of a chromosome may be “altered;” for instance the entire portion may be absent due to a deletion or may be rearranged (e.g., inversions, translocations, expanded or contracted due to changes in repeat regions). In the case of a deletion, an attempt to hybridize (i.e., specifically bind) a probe homologous to a particular portion of a chromosome could result in a negative result (i.e., the probe could not bind to the sample containing genetic material suspected of containing the missing portion of the chromosome). Thus, hybridization of a probe homologous to a particular portion of a chromosome may be used to detect alterations in a portion of a chromosome.

The term “sequences associated with a chromosome” as used herein, means preparations of chromosomes (e.g., spreads of metaphase chromosomes), nucleic acid extracted from a sample containing chromosomal DNA (e.g., preparations of genomic DNA); the RNA that is produced by transcription of genes located on a chromosome (e.g., hnRNA and mRNA), and cDNA copies of the RNA transcribed from the DNA located on a chromosome. Sequences associated with a chromosome may be detected by numerous techniques including probing of Southern and Northern blots and in situ hybridization to RNA, DNA, or metaphase chromosomes with probes containing sequences homologous to the nucleic acids in the above listed preparations.

The term “portion” as used herein, when in reference to a nucleotide sequence (as in “a portion of a given nucleotide sequence”), refers to fragments of that sequence. The fragments may range in size from four nucleotides to the entire nucleotide sequence minus one nucleotide (10 nucleotides, 20, 30, 40, 50, 100, 200, etc.).

The term “coding region” as used herein, when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets, which specify stop codons (i.e., TAA, TAG, TGA).

The term “purified” or “to purify” as used herein, refers to the removal of contaminants from a sample. For example, BSND antibodies are purified by removal of contaminating non-immunoglobulin proteins; they are also purified by the removal of immunoglobulin that does not bind BSND. The removal of non-immunoglobulin proteins and/or the removal of immunoglobulins that do not bind BSND results in an increase in the percent of BSND-reactive immunoglobulins in the sample. In another example, recombinant BSND polypeptides are expressed in bacterial host cells and the polypeptides are purified by the removal of host cell proteins; the percent of recombinant BSND polypeptides is thereby increased in the sample.

The term “recombinant DNA molecule” as used herein, refers to a DNA molecule that is comprised of segments of DNA joined together by means of molecular biological techniques.

The term “recombinant protein” or “recombinant polypeptide” as used herein, refers to a protein molecule that is expressed from a recombinant DNA molecule.

The term “native protein” as used herein, indicates that a protein does not contain amino acid residues encoded by vector sequences; that is the native protein contains only those amino acids found in the protein as it occurs in nature. A native protein may be produced by recombinant means or may be isolated from a naturally occurring source.

The term “portion” as used herein, when in reference to a protein (as in “a portion of a given protein”) refers to fragments of that protein. The fragments may range in size from four consecutive amino acid residues to the entire amino acid sequence minus one amino acid.

The term “Southern blot,” as used herein, refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. J. Sambrook et al., Molecular Cloning. A Laboratory Manual, Cold Spring Harbor Press, NY, pp 9.31-9.58 (1989).

The term “Northern blot,” as used herein, refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. J. Sambrook, et al., supra, pp 7.39-7.52 (1989).

The term “Western blot” as used herein, refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

The term “antigenic determinant” as used herein, refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transgene” as used herein, refers to a foreign, heterologous, or autologous gene that is placed into an organism by introducing the gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene. The term “autologous gene” is intended to encompass variants (e.g., polymorphisms or mutants) of the naturally occurring gene. The term transgene thus encompasses the replacement of the naturally occurring gene with a variant form of the gene.

The term “vector” as used herein, refers to nucleic acid molecules that transfer DNA segment(s) from one cell to another. The term “vehicle” is sometimes used interchangeably with “vector.”

The term “expression vector” as used herein, refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “host cell” as used herein, refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” as used herein, refer to levels of mRNA to indicate a level of expression approximately 2-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of mRNA present in the band corresponding in size to the correctly spliced PAD4 transgene RNA is quantified; other minor species of RNA which hybridize to the transgene probe are not considered in the quantification of the expression of the transgenic mRNA.

The term “transfection” as used herein, refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The terms “stable transfection” or “stably transfected” as used herein, refer to the introduction and integration of foreign DNA into the genome of the transfected cell. The term “stable transfectant” refers to a cell that has stably integrated foreign DNA into the genomic DNA.

The terms “transient transfection” or “transiently transfected” as used herein, refer to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” as used herein, refers to a technique for the introduction of nucleic acids into a cell. The uptake of nucleic acids by cells is enhanced when the nucleic acid is presented as a calcium phosphate-nucleic acid co-precipitate. Graham and van der Eb (Graham and van der Eb, Virol., 52:456 (1973).

The term “composition comprising a given polynucleotide sequence” as used herein, refers broadly to any composition containing the given polynucleotide sequence. The composition may comprise an aqueous solution. Compositions comprising polynucleotide sequences encoding a PAD4 amino acid sequence (e.g., SEQ ID NO: 1) or fragments thereof may be employed as hybridization probes. In this case, the PAD4 encoding polynucleotide sequences are typically employed in an aqueous solution containing salts (e.g., NaCl), detergents (e.g., SDS), and other components (e.g., Denhardt's solution, dry milk, salmon sperm DNA, etc.).

The term “test compound” as used herein, refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein, is used in its broadest sense. For example, a sample may be derived from a body fluid (i.e., for example, whole blood, blood serum, blood plasma, sweat, lymph fluid, bile fluid, urine, semen, mucosal secretions etc.) or from body tissues (i.e., for example, liver, kidney, breast, lung, prostate, brain etc.). Generally a tissue sample may be derived from a biopsy procedure. Alternatively, a sample may be obtained under laboratory conditions (i.e., for example, from an in vitro cell culture) or from an inanimate surface (i.e., for example, by a swab).

The term “response,” as used herein, when used in reference to an assay, refers to the generation of a detectable signal (e.g., accumulation of reporter protein, increase in ion concentration, accumulation of a detectable chemical product).

The term “reporter gene” as used herein, refers to a gene encoding a protein that may be assayed. Examples of reporter genes include, but are not limited to, luciferase (See, e.g., deWet et al., Mol. Cell. Biol. 7:725 [1987] and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from CLONTECH Laboratories, Palo Alto, Calif.), chloramphenicol acetyltransferase, β-galactosidase, alkaline phosphatase, and horse radish peroxidase.

The term “pharmaceutically acceptable” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “therapeutically effective amount” as used herein, with respect to a drug dosage, shall mean that dosage that provides the specific pharmacological response for which the drug is administered or delivered to a significant number of subjects in need of such treatment. It is emphasized that ‘therapeutically effective amount,’ administered to a particular subject in a particular instance will not always be effective in treating the diseases described herein, even though such dosage is deemed a “therapeutically effective amount” by those skilled in the art. Specific subjects may, in fact, be “refractory” to a “therapeutically effective amount”. For example, a refractory subject may have a low bioavailability such that clinical efficacy is not obtainable. It is to be further understood that drug dosages are, in particular instances, measured as oral dosages, or with reference to drug levels as measured in blood.

The term “symptom” as used herein, refers to any subjective, objective or quantitative evidence of a disease or other physical abnormality in a subject or patient. For example, a cancer symptom may include, but is not limited to, a tumor, pain, headache, nausea etc.

The term “symptom is reduced” as used herein, refers to a qualitative or quantitative reduction in detectable symptoms, including, but not limited to, a detectable impact on the rate of recovery from disease (e.g. rate of tumor regression) or a detectable impact on the rate of development of disease (e.g., rate of tumor growth).

The term “refractory” as used herein, refers to any subject that does not respond with an expected clinical efficacy following the delivery of a compound as normally observed by practicing medical personnel.

The term “delivering” or “administering” as used herein, refers to any route for providing a pharmaceutical or a nutraceutical to a subject as accepted as standard by the medical community. For example, the present invention contemplates routes of delivering or administering that include, but are not limited to, intratumoral, oral, transdermal, intravenous, intraperitoneal, intramuscular, or subcutaneous.

The term “subject” or “patient” as used herein, refers to any animal to which an embodiment of the present invention may be delivered or administered. For example, a subject may be a human, dog, cat, cow, pig, horse, mouse, rat, gerbil, hamster etc.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “cell” as used herein, refers to any small, usually microscopic, mass of protoplasm bounded externally by a semipermeable membrane, usually including one or more nuclei and various nonliving products, capable alone or interacting with other cells of performing all the fundamental functions of life, and forming the smallest structural unit of living matter capable of functioning independently. For example, a cell as contemplated herein includes, but is not limited to, an epithelial cell, a breast cell, a nerve cell, a liver cell, a lung cell, a kidney cell etc. Further, cells as contemplated herein may include, but are not limited to, normal cells (i.e., non-cancerous cells) or transformed cells (i.e., cancerous cells).

The term “population” as used herein, refers to any mixture of biological cells that are similar in physiology, biochemistry, and genetics. For example, a population of normal cells may comprise liver and/or kidney cells that exhibit no abberant phenotypes and/or growth disorders. Alternatively, a population of cancer cells may comprise liver and/or kidney cells that do exhibit abberant phenotypes and/or growth disorders. For example, a growth disorder may be characterized by uncontrolled proliferation of the population of cancer cells such that a tumor is formed.

### BRIEF DESCRIPTION OF THE FIGURES

FIG. 1 presents exemplary data of a calorimetric 96 well microtiter plate screen for citrulline production. The bright red wells are indicative of PAD4 variants having arginine deiminase activity.

FIG. 2 presents exemplary data showing a graph of PAD4 variant R639E exhibiting Michaelis kinetics with L-arg as a substrate (open circles), and no demonstrable activity against the peptidyl-arginine substrate analog benzoyl-L-arg (closed circles).

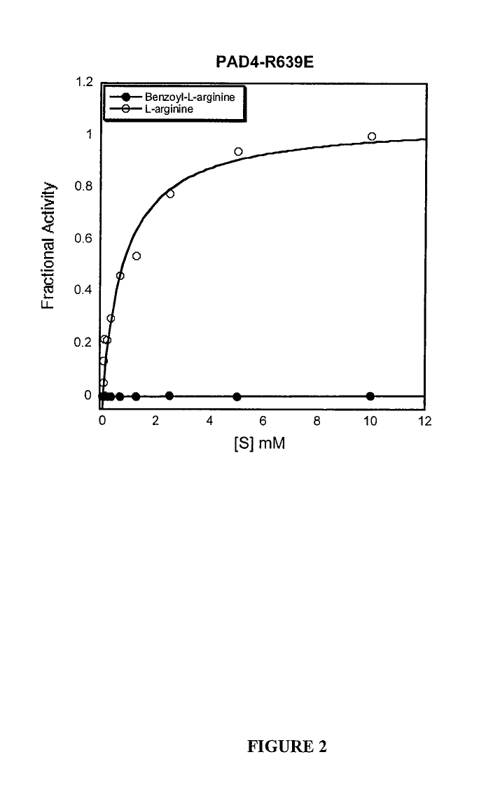

FIG. 3 presents one embodiment of a PAD4 wild type amino acid sequence (SEQ ID NO: 1).

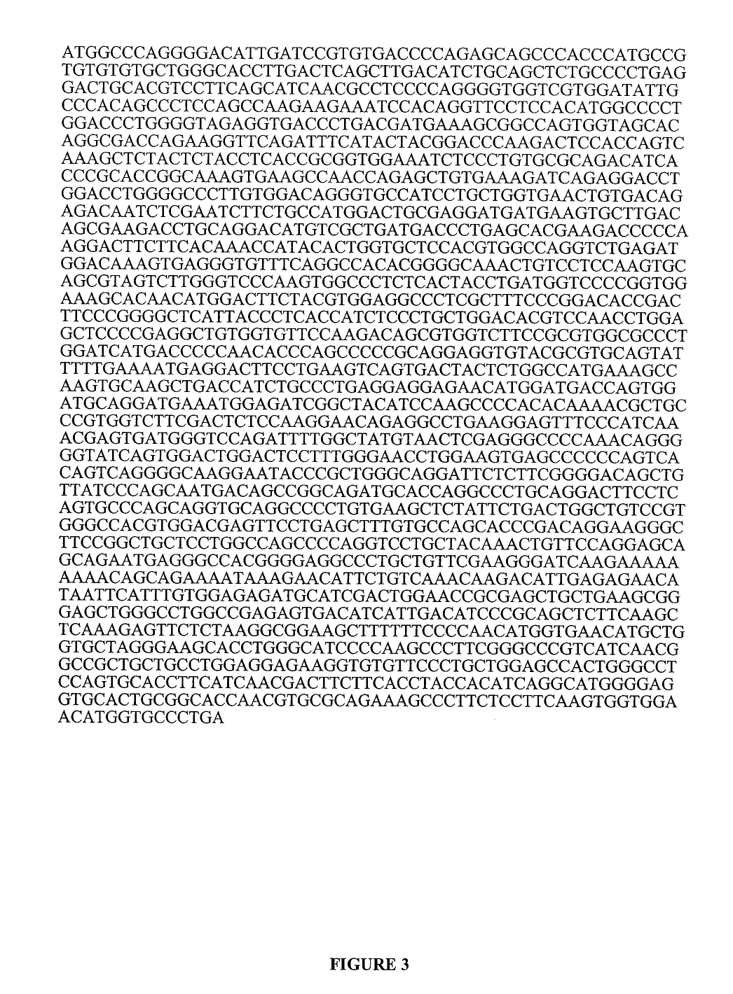

### DETAILED DESCRIPTION

This invention is related to compositions and methods for the treatment of cancer. In come embodiments, the invention contemplates human arginine degrading enzyme variants. For example. a rationally guided and directed evolution approach may be employed to create a humanized peptidyl arginine deiminase IV (PAD4) with arginine degrading activity.

### I. Hepatocellular Carcinoma (HCC)

Hepatic carcinoma requires the amino acid arginine for growth. Depletion of arginine has been shown to lead to tumor death. In humans, arginine is not an essential amino acid since many adult somatic cells can re-synthesize arginine from other sources. One method of arginine depletion can be effected via the action of enzymes that hydrolyze the amino acid. While human arginase enzymes do not have the properties required for the systemic depletion of arginine for therapeutic purposes, arginine deiminase, a bacterial enzyme from Mycopkasma hominus, has been shown to be therapeutically effective in the clinic and is currently being evaluated in a Phase II clinical trial. In addition, arginine deiminase treatment has been shown to cause remission of human melanomas.

The M. hominus bacterial arginase enzyme described above may be covalently linked to polyethylene glycol in order to improve serum half-life and reduce immunogenicity. Arginine deiminase, being a bacterial protein, is recognized as a foreign body by the human immune system and elicits an immune response in the form of specific antibodies. Anti-arginine deiminase antibodies can trigger adverse reactions in some cases and inhibit the catalytic activity and/or increase the clearance of the enzyme. Such adverse immune responses are not unique to arginine deiminase; other heterologous proteins including cancer therapeutic enzymes (e.g. asparaginase) are well documented to induce the formation of antibodies in patients in turn resulting in termination of therapy.

### II. Current Cancer Therapy Regimes

A. Enzymic Amino Acid Depletion

Some cancers may not be capable of synthesizing arginine. Consequently, amino-acid depletion (i.e., for example, arginine) has been proposed as a treatment of cancer, where malignant auxotrophic cells are essentially starved (1). For example, bacterial asparaginase, which catalyzes the conversion of asparagine to aspartate and ammonia, has been used clinically as a chemotherapeutic agent against acute lymphoblastic leukemia (ALL) and certain types of non-Hodgkin's lymphoma. Unfortunately, asparaginase has clinically relevant toxicity and immunogenicity (2).

Approximately 60% of high risk ALL patients develop neutralizing antibodies to therapeutic use of E. coli asparaginase (3). Patients developing an immune response to E. coli asparaginase may have the option to switch to an Erwinia species asparaginase. Attempts to reduced immunogenicity and extend serum half-life have been made by administering polyethylene glycol (PEG)-conjugated asparaginase. However, 20% of high risk ALL patients still develop antibodies against PEG-asparaginase (3).

Immunogenicity is a potential issue for any exogenous enzyme that is used as a human therapeutic agent. As the immune response to non-human enzyme therapeutics can be life threatening, technologies for developing enzymes that display the desired therapeutic catalytic activity without eliciting immune responses are highly desirable. One approach for attenuating harmful immune responses is to engineer enzymes with the desired activity by mutating a human enzyme. Properly performed this approach results in a protein whose sequence is >95% of human origin and which contains few or no novel epitopes that can elicit a dangerous immune response.

B. Arginine Depletion

Arginine is not an essential amino acid but malignant cancer cells appear to have a high demand for this particular amino acid (4). In normal cells, arginine may be synthesized in two steps: i) argininosuccinate synthetase (AS) converts citrulline and aspartate to argininosuccinate; and ii) argininosuccinate lyase (AL) conversion of argininosuccinate to arginine and fumarate. Further, melanomas and hepatocellular carcinomas (HCCs) have been shown to be auxotrophic for arginine, and Northern blots have revealed that argininosuccinate synthetase mRNA was undetectable in some carcinoma cell lines (5, 6). Recently, renal cell carcinomas (RCCs) were also found to be deficient in AS expression (7). Consequently, arginine depletion has been suggested as a potential chemotherapeutic strategy for auxotrophic melanomas including, but not limited to, HCCs and/or RCCs.

C. Arginine Deiminase Administration

The bacterial enzyme, arginine deiminase (ADI) (EC 3.5.3.6), which catalyses the hydrolysis of arginine to citrulline and ammonia, has been suggested as an anticancer agent. ADI has been observed to suppress growth in in vitro murine cell lines, and has prolonged in vivo mouse survival (8). ADI was also observed to inhibit the growth of fresh or cultured lymphatic leukemia cells (LLCs), however, LLCs are not arginine auxotrophs. (9) One suggested mechanism for these unexpected effects in LLCs is that ammonia is the therapeutic factor (released through ADI catalysis), rather than arginine depletion per se (10). While native ADI has been reported to inhibit growth of argininosuccinate synthetase-deficient melanomas and HCCs in vitro, appreciable inhibition of tumor growth using in vivo required large daily doses (5).

Improvements in ADI efficacy has been attempted by pegylation. For example, one mouse model study reported that ADI pegylation extended circulation half-life by over 30 fold, lowered the required dose of ADI, and depleted serum arginine levels below detectable levels for 6-8 days (11). One human clinical trial (N=24) treating metastatic melanoma reported a 25% positive response rate where pegylated ADI administered once a week depleted plasma arginine below detectable levels. Toxicity was also relatively low (i.e., grades 1 & 2). Pegylated ADI also raised the anti-ADI antibody titer, but none of the plasma samples obtained from the patients were reported to inhibit ADI in vitro (12). For comparison, other single chemotherapeutic agents have only shown a 15-20% response rate for metastatic melanoma (13). Another human clinical trial studied HCC patients (N=19) that were administered pegylated ADI. Plasma samples were not observed to inhibit ADI, but the antibody titer was raised in these patients, which parallels the observed decrease in plasma ADI concentration (14). Although these antibodies did not appear to neutralize ADI activity, it is possible that these antibodies may facilitate ADI clearance, thereby necessitating a more frequent dosing regimen.

In one embodiment, the present invention contemplates a humanized ADI having a significantly reduced immunogenic response, thereby reducing the titer of ADI-specific antibodies. In one embodiment, administration of humanized ADI in patients provides significantly improved therapeutic benefits as compared to bacterial ADI.

### III. Protein Humanization

A. Humanized Antibodies

Antibody humanization is generally believed to have greatly improved antibody therapeutics. In fact, most therapeutic antibodies approved by the United States Food & Drug Administration are either humanized, or fully human, proteins and exhibit far superior immunogenicity profiles relative to comparable mouse antibodies.

The humanization of non-antibody proteins (i.e., for example, an enzyme such as ADI) is not compatible with the general procedures that are used to create humanized antibodies. For example, the non-antibody protein humanization is highly sensitive to the replacement of large sequence segments with homologous sequences from other species. Antibodies are generally modular and contain conserved sequences, whereas non-antibody proteins are highly diverse and contain many unique sequences responsible for non-antibody protein activity. Consequently, the humanization of an enzyme is a highly empirical process.

In one embodiment, the present invention contemplates a method for generating bacterially derived ADI enzymes comprising >95% human amino acid sequence.

B. Humanized ADI Enzyme

Humans are believed to have at least five PAD isozymes (i.e., for example, PAD1-4, and 6) that utilize peptidyl arginine as a substrate and may be dependent on Ca²⁺ ion for activity. The PAD4 Ca²⁺ requirement was determined using small peptide-like arginine analogs where the K_{0.5} was measured in the mid-micromolar range (19). Since the serum [Ca²⁺] is in the range of 1-1.5 mM a major fraction of an engineered PAD4 should be active in vivo in the bloodstream. PAD is easily expressed in E. coli thereby facilitating mutagenesis and selection for altering substrate specificity (infra).

Although it is not necessary to understand the mechanism of an invention, it is believed that PADs are multidomain enzymes with two immunoglobulin-like N-terminal domains and a catalytic C-terminal domain that is structurally conserved with the other members of this superfamily. For example, these isoforms may have different tissue distributions and are believed to citrullinate substrate proteins including, but not limited to, keratins, myelin basic protein, filagrin, histone, and fibrins (15).

PAD isoform protein substrate specificities are not well defined. PADs have been implicated in certain diseases such as rheumatoid arthritis and multiple sclerosis, where the generation of autoantibodies against citrullinated proteins such as fibrin and myelin basin protein have been reported (16). Some studies suggest that PAD may be a drug target and susceptible to small molecule inhibitors (17, 18).

In one embodiment, the present invention contemplates a method comprising directed evolution to create a humanized arginine deiminase from a bacterial PAD4 enzyme. In one embodiment, the PAD4 is a fast enzyme comprising a kcat of 4-6 s⁻¹. Although it is not necessary to understand the mechanism of an invention it is believed that a fast enzyme PAD4 hydrolyzes arginine rapidly thereby allowing the administration of low doses to provide a therapeutic effect with minimal side effects (i.e., for example, passive immunization).

PAD4-bound substrate complex crystal structures have been reported. Comparisons of structural overlays between PAD4 and ADIs show that the respective residues involved in catalysis and/or binding the guanidine moiety of arginine are highly conserved. In both PAD and ADI, the carboxyl residues of Asp³⁵⁰ and Asp⁴⁷³ (utilizing PAD4 numbering) coordinate the substrates guanidino nitrogens. In both enzymes, substrates are cleaved between the conserved Cys⁶⁴⁵ and His⁴⁷¹ residues. Although it is not necessary to understand the mechanism of an invention it is believed that Cys⁶⁴⁵ is an active site nucleophile, mounting an attack on the guanidino carbon thereby forming a covalent thiourea intermediate with a concomitant loss of ammonia. It is further believed that His⁴⁷¹ acts as a general acid during formation of the covalent intermediate and then as a general base in creating a hydroxide ion for attack and hydrolysis of the intermediate. PAD and ADI may have structural differences where: i) the peptidyl-amide bond of PAD's protein substrate binds; ii) the free amino/carboxy termini of L-arg bind in ADI; iii) PAD4's active site is open, thereby allowing access to its protein substrates; and iv) ADI has an extra loop that closes down upon the active site when substrate binds.

In one embodiment, the present invention contemplates a method comprising mutagenizing a wild type PAD enzyme thereby converting catalytic activity to free arginine. In one embodiment, the mutant PAD enzyme comprises catalytic activity to arginine but not to peptidyl arginine, which is the substrate hydrolyzed by the wild type PAD4 enzyme. In one embodiment, the mutagenizing comprises structure guided mutagenesis. In one embodiment, the mutagenizing comprises random mutagenesis. In one embodiment, the method further comprises a high throughput arginine deiminase activity assay.

### IV. Directed Evolution

Directed evolution experimentally modifies a biological molecule towards a desirable property, and can be achieved by mutagenizing one or more parental molecular templates and identifying any desirable molecules among the progeny molecules. Several currently available technologies are available.

Molecular mutagenesis occurs in nature and has resulted in the generation of a wealth of biological compounds that have shown utility in certain industrial applications. However, evolution in nature often selects for molecular properties that are discordant with many unmet industrial needs. Additionally, it is often the case that when an industrially useful mutation would otherwise be favored at the molecular level, natural evolution often overrides the positive selection of such mutations when there is a concurrent detriment to an organism as a whole (such as when a favorable mutation is accompanied by a detrimental mutation). Additionally still, natural evolution is slow, and places high emphasis on fidelity in replication. Finally, natural evolution prefers a path paved mainly by beneficial mutations while tending to avoid a plurality of successive negative mutations, even though such negative mutations may prove beneficial when combined, or may lead—through a circuitous route—to final state that is beneficial.

Directed evolution, on the other hand, can be performed much more rapidly and aimed directly at evolving a molecular property that is industrially desirable where nature does not provide one. An exceedingly large number of possibilities exist for purposeful and random combinations of amino acids within a protein to produce useful hybrid proteins and their corresponding biological molecules encoding for these hybrid proteins, i.e., DNA, RNA. Accordingly, there is a need to produce and screen a wide variety of such hybrid proteins for a desirable utility, particularly widely varying random proteins.

The complexity of an active sequence of a biological macromolecule (e.g., polynucleotides, polypeptides, and molecules that are comprised of both polynucleotide and polypeptide sequences) has been called its information content (“IC”), which has been defined as the resistance of the active protein to amino acid sequence variation (calculated from the minimum number of invariable amino acids (bits) required to describe a family of related sequences with the same function). Proteins that are more sensitive to random mutagenesis have a high information content.

Molecular biology developments, such as molecular libraries, provide ways to select functional sequences from random libraries. In such libraries, most residues can be varied (although typically not all at the same time) depending on compensating changes in the context. Thus, while a 100 amino acid protein can contain only 2,000 different mutations, 20 sup. 100 sequence combinations are possible.

Information density is the IC per unit length of a sequence. Active sites of enzymes tend to have a high information density. By contrast, flexible linkers of information in enzymes have a low information density.

Current methods in widespread use for creating alternative proteins in a library format include, but are not limited to, error-prone polymerase chain reactions, oligonucleotide-directed mutagenesis, and cassette mutagenesis, in which the specific region to be optimized is replaced with a synthetically mutagenized oligonucleotide. In both cases, a substantial number of mutant sites are generated around certain sites in the original sequence.

In nature, the evolution of most organisms occurs by natural selection and sexual reproduction. Sexual reproduction ensures mixing and combining of the genes in the offspring of the selected individuals. During meiosis, homologous chromosomes from the parents line up with one another and cross-over part way along their length, thus randomly swapping genetic material. Such swapping or shuffling of the DNA allows organisms to evolve more rapidly.

In recombination, because the inserted sequences were of proven utility in a homologous environment, the inserted sequences are likely to still have substantial information content once they are inserted into the new sequence.

Theoretically there are 2,000 different single mutants of a 100 amino acid protein. However, a protein of 100 amino acids has 20¹⁰⁰ possible sequence combinations, a number which is too large to exhaustively explore by conventional methods. It would be advantageous to use a system which allows generation and screening of all of these possible combination mutations.

A. Error Prone Polymerase Chain Reaction

In some embodiments, directed evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5. Moore and Arnold, Nat. Biotech., 14, 458 (1996); Leung et al., Technique, 1:11 (1989); Eckert and Kunkel, PCR Methods Appl., 1:17-24 (1991); Caldwell and Joyce, PCR Methods Appl., 2:28 (1992); and Zhao and Arnold, Nuc. Acids. Res., 25:1307 (1997). After mutagenesis, the resulting clones are selected for desirable activity (e.g., screened for enzymatic activity). Successive rounds of mutagenesis and selection are often necessary to develop enzymes with desirable properties. It should be noted that only the useful mutations are carried over to the next round of mutagenesis.

B. Amino Acid Randomization

In some embodiments, directed evolution is performed by amino acid randomization. One randomization method for rapidly and efficiently producing a large number of alterations in a known amino acid sequence or for generating a diverse population of variable or random sequences is known as codon-based synthesis or mutagenesis. U.S. Pat. Nos. 5,264,563 and 5,523,388 (both herein incorporated by reference); and Glaser et al. J. Immunology 149:3903 (1992). Briefly, coupling reactions for the randomization of, for example, all twenty codons which specify the amino acids of the genetic code are performed in separate reaction vessels and randomization for a particular codon position occurs by mixing the products of each of the reaction vessels. Following mixing, the randomized reaction products corresponding to codons encoding an equal mixture of all twenty amino acids are then divided into separate reaction vessels for the synthesis of each randomized codon at the next position. For the synthesis of equal frequencies of all twenty amino acids, up to two codons can be synthesized in each reaction vessel.

Variations to these synthesis methods also exist and include for example, the synthesis of predetermined codons at desired positions and the biased synthesis of a predetermined sequence at one or more codon positions. Biased synthesis involves the use of two reaction vessels where the predetermined or parent codon is synthesized in one vessel and the random codon sequence is synthesized in the second vessel. The second vessel can be divided into multiple reaction vessels such as that described above for the synthesis of codons specifying totally random amino acids at a particular position. Alternatively, a population of degenerate codons can be synthesized in the second reaction vessel such as through the coupling of NNG/T nucleotides where N is a mixture of all four nucleotides. Following synthesis of the predetermined and random codons, the reaction products in each of the two reaction vessels are mixed and then redivided into an additional two vessels for synthesis at the next codon position.

A modification to the above-described codon-based synthesis for producing a diverse number of variant sequences can similarly be employed for the production of the variant populations described herein. This modification is based on the two vessel method described above which biases synthesis toward the parent sequence and allows the user to separate the variants into populations containing a specified number of codon positions that have random codon changes.

Briefly, this synthesis is performed by continuing to divide the reaction vessels after the synthesis of each codon position into two new vessels. After the division, the reaction products from each consecutive pair of reaction vessels, starting with the second vessel, is mixed. This mixing brings together the reaction products having the same number of codon positions with random changes. Synthesis proceeds by then dividing the products of the first and last vessel and the newly mixed products from each consecutive pair of reaction vessels and redividing into two new vessels. In one of the new vessels, the parent codon is synthesized and in the second vessel, the random codon is synthesized. For example, synthesis at the first codon position entails synthesis of the parent codon in one reaction vessel and synthesis of a random codon in the second reaction vessel. For synthesis at the second codon position, each of the first two reaction vessels is divided into two vessels yielding two pairs of vessels. For each pair, a parent codon is synthesized in one of the vessels and a random codon is synthesized in the second vessel. When arranged linearly, the reaction products in the second and third vessels are mixed to bring together those products having random codon sequences at single codon positions. This mixing also reduces the product populations to three, which are the starting populations for the next round of synthesis. Similarly, for the third, fourth and each remaining position, each reaction product population for the preceding position are divided and a parent and random codon synthesized.

Following the above modification of codon-based synthesis, populations containing random codon changes at one, two, three and four positions as well as others can be conveniently separated out and used based on the need of the individual. Moreover, this synthesis scheme also allows enrichment of the populations for the randomized sequences over the parent sequence since the vessel containing only the parent sequence synthesis is similarly separated out from the random codon synthesis.

Other methods for producing a large number of alterations in a known amino acid sequence or for generating a diverse population of variable or random sequences include, for example, degenerate or partially degenerate oligonucleotide synthesis. Codons specifying equal mixtures of all four nucleotide monomers, represented as NNN, results in degenerate synthesis. Whereas partially degenerate synthesis can be accomplished using, for example, the NNG/T codon described previously. Other methods can alternatively be used including, but not limited to, the use of statistically predetermined, or variegated, codon synthesis. U.S. Pat. Nos. 5,223,409 and 5,403,484 (both herein incorporated by reference).

Once the populations of altered variable region encoding nucleic acids have been constructed as described above, they can be expressed to generate a population of altered variable region polypeptides that can be screened for binding affinity. For example, the altered variable region encoding nucleic acids can be cloned into an appropriate vector for propagation, manipulation and expression. Such vectors should contain all expression elements sufficient for the transcription, translation, regulation, and if desired, sorting and secretion of the altered variable region polypeptides. The vectors also can be for use in either prokaryotic or eukaryotic host systems so long as the expression and regulatory elements are of compatible origin. The expression vectors can additionally included regulatory elements for inducible or cell type-specific expression. Many host systems are compatible with particular vectors which comprise regulatory or functional elements sufficient to achieve expression of the polypeptides in soluble, secreted or cell surface forms.

Appropriate host cells include, but are not limited to, bacteria and corresponding bacteriophage expression systems, yeast, avian, insect and mammalian cells. Methods for recombinant expression, screening and purification of populations of altered variable regions or altered variable region polypeptides within such populations in various host systems have been reported, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998). The choice of a particular vector and host system for expression and screening of altered variable regions depend on the preference of the user.

The expressed population of altered variable region polypeptides can be screened for the identification of one or more altered variable region species exhibiting binding affinity substantially the same or greater than the wild type variable region. Screening can be accomplished using various methods for determining the binding affinity of a polypeptide or compound. Additionally, methods based on determining the relative affinity of binding molecules to their partner by comparing the amount of binding between the altered variable region polypeptides and the wild type variable region can similarly be used for the identification of species exhibiting binding affinity substantially the same or greater than the wild type variable region. All of such methods can be performed, for example, in solution or in solid phase. Moreover, various formats of binding assays include, but are not limited to, immobilization to filters such as nylon or nitrocellulose; two-dimensional arrays, enzyme linked immunosorbant assay (ELISA), radioimmune assay (RIA), panning and plasmon resonance. Such methods can be found described in, for example, Sambrook et al., supra, and Ansubel et al. For the screening of populations of polypeptides such as the altered variable region populations produced by the methods of the invention, immobilization of the populations of altered variable regions to filters or other solid substrate is particularly advantageous because large numbers of different species can be efficiently screened for antigen binding. Such filter lifts will allow for the identification of altered variable regions that exhibit substantially the same or greater binding affinity compared to the wild type variable region. Alternatively, if the populations of altered variable regions are expressed on the surface of a cell or bacteriophage, for example, panning on immobilized substrate can be used to efficiently screen for the relative binding affinity of species within the population and for those which exhibit substantially the same or greater binding affinity than the wild type variable region.

Another affinity method for screening populations of altered variable region polypeptides is a capture lift assay that is useful for identifying a binding molecule having selective affinity for a ligand (Watkins et. al., (1997)). This method employs the selective immobilization of altered variable regions to a solid support and then screening of the selectively immobilized altered variable regions for selective binding interactions against the cognate antigen or binding partner. Selective immobilization functions to increase the sensitivity of the binding interaction being measured since initial immobilization of a population of altered variable regions onto a solid support reduces non-specific binding interactions with irrelevant molecules or contaminants which can be present in the reaction.

Another method for screening populations or for measuring the affinity of individual altered variable region polypeptides is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (kₒₙ) and disassociation rates (k_{off}).

Measurements of kₒₙ and k_{off} values can be advantageous because they can identify altered variable regions or optimized variable regions that are therapeutically more efficacious. For example, an altered variable region, or monomeric binding fragment thereof, can be more efficacious because it has, for example, a higher kₒₙ value compared to variable regions and monomeric binding fragments that exhibit similar binding affinity. Increased efficacy is conferred because molecules with higher kₒₙ values can specifically bind and inhibit their target at a faster rate. Similarly, a molecule of the invention can be more efficacious because it exhibits a lower k_{off} value compared to molecules having similar binding affinity. Increased efficacy observed with molecules having lower k_{off} rates can be observed because, once bound, the molecules are slower to dissociate from their target. Although described with reference to the altered variable regions and optimized variable regions of the invention including, but not limited to, monomeric variable region binding fragments thereof, the methods described above for measuring associating and disassociation rates are applicable to essentially any peptide, protein, or fragment thereof for identifying more effective binders for therapeutic or diagnostic purposes.

Methods for measuring the affinity, including association and disassociation rates using surface plasmon can be found described in, for example, Jonsson and Malmquist, Advances in Biosensors, 2:291 336 (1992) and Wu et al. Proc. Natl. Acad. Sci. USA, 95:6037 6042 (1998). Moreover, one apparatus for measuring binding interactions is a BIAcore 2000 instrument which is commercially available through Pharmacia Biosensor, (Uppsala, Sweden).

Using any of the above described screening methods, as well as others, an altered variable region having binding affinity substantially the same or greater than the wild type variable region is identified by detecting the binding of at least one altered variable region within the population to its antigen or cognate ligand. Additionally, the above methods can alternatively be modified by, for example, the addition of substrate and reactants, to identify using the methods of the invention, altered variable regions having catalytic activity substantially the same or greater that the wild type variable region within the populations. Comparison, either independently or simultaneously in the same screen, with the wild type variable region will identify those binders that have substantially the same or greater binding affinity as the wild type.

Detection methods for identification of binding species within the population of altered variable regions can be direct or indirect and can include, for example, the measurement of light emission, radioisotopes, calorimetric dyes and fluorochromes. Direct detection includes methods that operate without intermediates or secondary measuring procedures to assess the amount of bound antigen or ligand. Such methods generally employ ligands that are themselves labeled by, for example, radioactive, light emitting or fluorescent moieties. In contrast, indirect detection includes methods that operate through an intermediate or secondary measuring procedure. These methods generally employ molecules that specifically react with the antigen or ligand and can themselves be directly labeled or detected by a secondary reagent. For example, an enzyme specific for a substrate can be detected using a secondary antibody capable of interacting with the first antibody specific for the substrate, again using the detection methods described above for direct detection. Indirect methods can additionally employ detection by enzymatic labels. Moreover, for the specific example of screening for catalytic proteins (i.e., for example, an enzyme), the disappearance of a substrate or the appearance of a product can be used as an indirect measure of binding affinity or catalytic activity.

In one embodiment, the present invention contemplates a method for simultaneously grafting and optimizing the catalytic activity of a protein fragment. The method consists of: (a) constructing a population of altered enzyme variable region encoding nucleic acids; (b) expressing the population variable region encoding nucleic acids to produce diverse combinations of monomeric variable region binding fragments, and (c) identifying one or more monomeric variable regions having activity substantially the same or greater than the wild type variable region enzyme.

The invention additionally provides a method of optimizing the activity of an enzyme. This method comprises: (a) constructing a population of protein variable region encoding nucleic acids, said population comprising a plurality of different amino acids at one or more amino acid residue positions; (b) expressing said population of variable region encoding nucleic acids, and (c) identifying one or more variagated regions having activity substantially the same or greater than the wild type enzyme.

Moreover, by incorporating variagated amino acid residues in two or more amino acid residue positions this method modifies catalytic activity and is therefore useful for simultaneously optimizing the binding affinity or catalytic activity of a protein and/or enzyme. Employing the methods for simultaneously grafting and optimizing, or for optimizing, it is possible to generate enzymes having increased catalytic activity as compared to the wild type enzyme.

Additionally, the methods described herein for optimizing are also are applicable for producing catalytic variable region fragments or for optimizing their catalytic activity. Catalytic activity can be optimized by changing, for example, the on or off rate, the substrate binding affinity, the transition state binding affinity, the turnover rate (kcat) or the Km. Amino acid residues selected for alteration are typically amino positions predicted to be relatively important for structure or function. Criteria that can be used for identifying amino positions to be altered include, for example, conservation of amino acids among polypeptide subfamily members and knowledge that particular amino acids are predicted to be important in polypeptide conformation or structure, as described above. Alternatively, potentially important amino acid residues can be characterized without structural information by synthesizing and expressing a combinatorial peptide library that contains all possible combinations of amino acids in the residue positions to be optimized.

The invention provides a method for identifying one or more functional amino acid positions of a polypeptide. The method consists of (a) constructing a population of nucleic acids encoding a population of altered polypeptides containing substitutions of one or more amino acid positions within a polypeptide; (b) expressing the population of nucleic acids; (c) identifying nucleic acids encoding altered polypeptides having a functional activity of the polypeptide; (d) sequencing a subset of nucleic acids encoding altered polypeptides having a functional activity, and (e) comparing an amino acid position in a polypeptide corresponding to an amino acid position in the subset of altered polypeptides wherein an amino acid position exhibiting a biased representation of amino acid residues indicates a functional amino acid position in the polypeptide.

The method of the invention directed to identifying a functional amino acid position in a polypeptide involves substituting one or more amino acid positions in a polypeptide with a plurality of amino acid residues, as described previously for optimizing the catalytic activity of an enzyme, and identifying altered polypeptides having an activity that is substantially the same or greater than the parent polypeptide. Functional amino acid positions identified using the methods of the invention are amino acid positions important for a conformation, functional activity or structure of a polypeptide. Functional activities of a polypeptide can include, for example, binding affinity to a substrate, ligand, or other interacting molecule, and catalytic activity.

The identification of functional amino acid positions in a polypeptide involves constructing a population of nucleic acids encoding a population of altered polypeptides containing amino acid substitutions at specific amino acid positions. Substituted amino acids include all twenty naturally occurring amino acid residues or a subset of amino acid residues, as described previously in detail. Nucleic acid populations can be constructed by any method as described previously. A population of nucleic acids encoding altered polypeptides is expressed in an appropriate host cell, and a functional activity of altered polypeptides is detected and compared with that of the polypeptide. Many methods are appropriate for determining a polypeptide functional activity can be used to compare polypeptide and altered polypeptide functional activities.

A subset of nucleic acids encoding altered polypeptides having a functional activity that is substantially the same or greater than that of the polypeptide is sequenced. A subset can include a few molecules to many members constituting the population of nucleic acids encoding altered polypeptides. For example, a subset can consist of about 2-5, 6-10, 10-20, and 21 or greater members of the population. The actual number sequenced will vary with the total size of the nucleic acid population. Generally, however, a subset of about 15-25 and typically about 20 members is sufficient in order to identify functional amino acids.

Amino acid residues at substituted positions in the polypeptide are compared to the corresponding position in altered polypeptides. An amino acid position that contains the same amino acid or a conservative substitution among the population of altered polypeptides exhibits biased representation of that amino acid residue. Biased representation indicates that a particular amino acid is required for polypeptide function. Amino acid positions that are biased are therefore considered important for functional activity of a polypeptide. Amino acid positions that contain a variety of substituted amino acids are unbiased and considered not important or less important for a polypeptide function.

The method of identifying an amino acid position important for polypeptide function is useful for a variety of applications, such as, for example, the determination of a consensus sequence of amino acids important for a polypeptide functional activity. A consensus sequence is useful for the optimization of a polypeptide function because amino acid positions determined to be important for functional activity can be unaltered while amino acid positions not important for activity can be varied. Polypeptide functions that can be optimized using the method of the invention include, for example, catalytic activity, polypeptide conformation and binding affinity.

The identification of a functional amino acid position in a polypeptide can be applied to determining a consensus sequence of amino acids that impart a particular activity to a polypeptide. For example, a consensus sequence that provides a catalytic activity to an enzyme can be determined using the methods of the invention. To identify amino acid positions that are important or critical to catalytic activity of an enzyme, one or more of amino acid positions are substituted with a plurality of amino acid substitutions, as described previously. A nucleic acid population encoding altered enzyme polypeptides is constructed and expressed in host cells. The catalytic activity of altered enzymes is measured and compared with a parent enzyme or other catalytically active form of the enzyme.

Nucleic acids encoding a subset of altered enzyme polypeptides identified by functional activity are sequenced, and the amino acid sequences of altered polypeptides are compared. Amino acid positions that contain a particular amino acid or a conservative substitution are determined to be important for a catalytic activity of the enzyme. A sequence of amino acids determined to be biased in a polypeptide can thus provide a consensus sequence that defines amino acid positions required for catalytic activity. A consensus sequence of residues important for various aspects of catalytic activity such as, for example, substrate binding, proper active site conformation, and co-factor binding can be identified using the methods of the invention by measuring enzyme catalytic activity, as described above.

Similarly, a consensus sequence associated with a particular conformation of a polypeptide can be determined using the method of the invention in essentially the same manner as described above for polypeptide catalytic activity. The amino acid positions that have functional roles in a polypeptide conformation can be determined so long as a particular conformation state can be detected and compared between a polypeptide and an altered polypeptide. For example, a consensus sequence of a polypeptide conformation that confers a particular functional activity to a polypeptide or a particular structural feature to a polypeptide can be determined using the methods of the invention. A structural feature can include, for example, the exposure of a certain amino acid on the surface of a polypeptide.

A consensus sequence of amino acid positions in a polypeptide important for catalytic activity can also be determined using the methods of the invention. For example, a consensus sequence for the activity of an enzyme with a substrate can be determined, and can be applied to the process of enzyme humanization.

The identification of a functional amino acid position in a polypeptide can be applied to determining the consensus sequence for a humanized version of an enzyme that preserves similar binding activity of the parent enzyme. For example, a library containing all possible combinations of human template and non-human parent enzyme residues in a selected number of amino positions can be synthesized by, for example, using codon-based mutagenesis. Polypeptides containing amino acid substitutions can then be screened by functional activity assays to identify altered polypeptides that have catalytic properties similar as the parent enzyme. Of the amino acid positions altered, only a small percentage of amino acid residue positions are typically critical for activity. Therefore, either a low or high throughput screening methods of identifying active humanized enzyme variants are compatible with the present invention. Sequencing of nucleic acids encoding humanized enzymes displaying a functional activity of the parent enzyme is then used to identify altered polypeptides. Thus, a consensus humanization sequence for maintaining full binding activity of an enzyme can be prepared by using bacterial enzymes grafted onto a human template on which amino acid positions are changed to the corresponding residue determined to be important for activity.

C. Gene Shuffling

In some embodiments, directed evolution comprises gene shuffling. For example, the polynucleotides of the present invention may be used in gene shuffling or sexual PCR procedures. Smith, Nature, 370:324 (1994); and U.S. Pat. Nos. 5,837,458; 5,830,721; 5,811,238; 5,733,731; (all of which are herein incorporated by reference). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DNaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes. Stemmer, Nature, 370:398 (1994); Stemmer, Proc. Natl. Acad. Sci. USA, 91:10747 (1994); Crameri et al., Nat. Biotech., 14:315 (1996); Zhang et al., Proc. Natl. Acad. Sci. USA, 94:4504 (1997); and Crameri et al., Nat. Biotech., 15:436 [1997]). Protein variants produced by directed evolution can be screened for enzymatic activity by the methods described herein.

A wide range of techniques are known for screening gene products of combinatorial libraries made by point mutations, and for screening cDNA libraries for gene products having a certain property. Such techniques will be generally adaptable for rapid screening of the gene libraries generated by the combinatorial mutagenesis or recombination of protein homologs or variants. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

### V. Pharmaceutical Compositions

The present invention further provides pharmaceutical compositions (e.g., comprising the polypeptides described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more polypeptide compounds (i.e., for example, a mutated PAD4) and (b) one or more conventional chemotherapeutic agents. Examples of such conventional chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### EXPERIMENTAL

The following examples illustrate some embodiments of PAD mutants exhibiting arginine deiminase activity that could be employed for human therapy (i.e., for example, to treat various carcinomas). These examples are not intended to be limiting and only provide one having ordinary skill in the art guidance to understand and utilize the invention.

### Example I

### 96-Well Plate Screen for ADI Activity and Ranking Clones

This example describes a colorimetric 96-well plate arginine deiminase activity assay by detecting the L-citrulline reaction. Clones displaying ADI activity as measured by this assay are then selected for further characterization. A library of PAD mutants can be constructed using any of a variety of techniques including, but not limited to, oligonucleotide mutagenesis, or error prone PCR DNA shuffling.

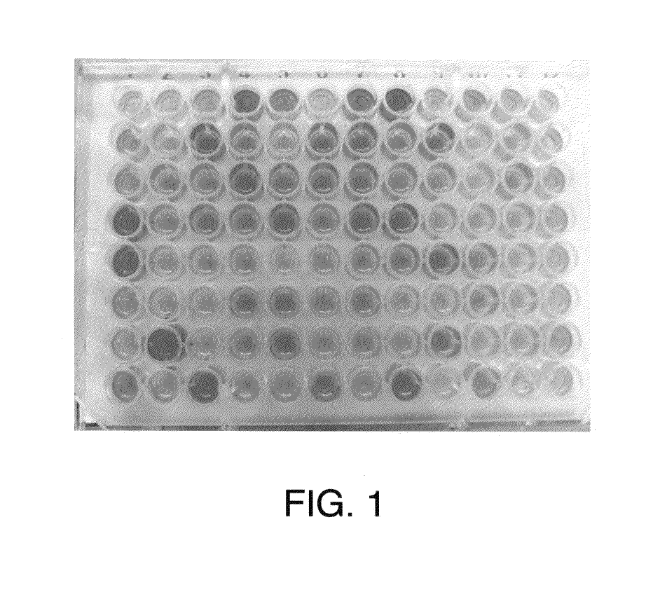

Single colonies are inoculated into 96-well culture plates containing 75 μL of TB media/well supplemented with 34 μg/ml chloramphenicol and 100 μg/ml ampicillin. Cells are then grown at 37° C. on a plate shaker until reaching an OD₆₀₀ of 0.8-1, then they are cooled to 25° C., whereupon an additional 75 μL of media containing 34 μg/ml chloramphenicol, 100 μg/ml ampicillin and 0.5 mM IPTG is added. Protein expression is performed by first growing the cells at 25° C. with shaking for 2-3 hrs after induction, and then transferring 100 μL of culture/well to a 96 well assay plate. The assay plates are then centrifuged to pellet the cells, the media is removed, and the cells are lysed by addition of 50 μL/well of B-PER protein extraction reagent (Pierce). An additional 50 μL/well of ˜2 mM L-Arg, 10 mM CaCl₂, and 5 mM dithiothreitol in a 100 mM Tris buffer, pH 7.6 is subsequently added and allowed to react at 37° C. After reacting 3-4 hrs, 100 μL/well of color developing reagent is added and the plate processed as described elsewhere (20). Colonies having the ability to produce L-citrulline result in formation of a bright red dye with a λₘₐₓ of 530 nm. See, FIG. 1.

### Example II

### PAD4 Saturation Mutagenesis Library of Residues Arg³⁷⁴ and Arg⁶³⁹

This example presents one embodiment showing a method to mutagenize a PAD4 enzyme.

Structural analysis of PAD4 shows that amino acids Arg³⁷⁴ and Arg⁶³⁹ appear to be involved in binding PAD's wild type substrate via a peptidyl-amide bond. A saturation library of PAD4 (i.e., for example, ˜10³ variants) was constructed by overlap extension polymerase chain reaction (PCR) using oligonucleotides with NNS randomized codons at positions corresponding to Arg³⁷⁴ and Arg⁶³⁹. The amplified DNA was ligated into a pGEX-6p1 vector and transformed into E. coli cells using standard techniques. Approximately 4000 clones were screened and those having increased ADI activity were identified. Plasmid DNA was then isolated from the ADI-positive E. coli clones and sequenced to identify the amino acid mutations conferring the improved ADI activity.

Specifically, a PAD4 library was constructed by overlap extension PCR using the following oligonucleotides: 5′-GGGCTGGCAAGCCACGTTTGGTG-3′ complementary to the 5′ region of the pGEX-6p1 vector, 5′-TTGGTACCGAATTCGCGGCCGCGAGCTCTTGC TTGCC-3′ complementary to the 3′ untranslated region of the PAD4 gene and containing a Not I restriction site (underlined), 5′-gactctccaaggaacNNSggcctgaaggagtttccc-3′ and 5′-AAACTCCTTCAGGCCSNNGTTCCTTGGAGAGTCGAAG-3′ to introduce random codons at the position corresponding to Arg³⁷⁴, and 5′-cttcttcacctaccacatcNNScatggggagg-3′ and 5′-CCCCATGSNNGATGTGGTAGGTGAAGAAG-3′ to introduce random codons at the position corresponding to Arg⁶³⁹.

The PAD4 gene with randomized codons at positions Arg³⁷⁴ and Arg⁶³⁹ was digested with EcoRI and Not I thereby allowing ligation into a pGEX-6p1 vector (Amersham Biosciences, Piscataway, N.J.) cut with the same restriction enzymes. The ligation mixture was transformed into DH5α E. coli cells. The transfected E. coli cells were then plated on LB-ampicillin plates and ˜8000 individual colonies were obtained. The plates were then scraped and mini-prepped to collect the library DNA. The plasmid DNA was then transformed into Rosetta-2 E. coli cells and plated on LB plates containing 34 μg/ml chloramphenicol and 100 g/ml ampicillin for subsequent screening in accordance with Example I.

The amino acid coding sequences at amino acid (AA) positions AA³⁷⁴ and AA⁶³⁹ were compared between clones selected at random versus those identified during the screening process. See, Table 1.

TABLE 1
Random selection versus screening identification of amino acid
coding found using PAD-R³⁷⁴/R⁶³⁹ library (parentheses
indicate encoded amino acid).

| Amino Acid Position | | | Amino Acid Position | | |
|---|---|---|---|---|---|
| Random | | | Screened | | |
| Selection | AA³⁷⁴ | AA⁶³⁹ | Selection | AA³⁷⁴ | AA⁶³⁹ |
| WT | AGA (R) | AGG (R) | WT | AGA (R) | AGG (R) |
| 1 | AAG (K) | TTG (L) | 1 | AGA (R) | AGG (R) |
| 2 | AGA (R) | AAC (N) | 2 | AAG (K) | CAC (H) |
| 3 | AGC (S) | TCC (S) | 3 | ATG (M) | GAG (E) |
| 4 | CCG (P) | TCC (S) | 4 | CGC (R) | CAC (H) |
| 5 | CGT (R) | CGC (R) | 5 | CGC (R) | AAC (N) |
| 6 | TCC (S) | AGG (R) | 6 | CGC (R) | GAG (E) |

### Example III

### PAD4 Saturation Mutagenesis Library of Residues Arg⁶³⁹ and His⁶⁴⁰

This example presents one embodiment showing a method to mutagenize a PAD4 enzyme. Transfected E. coli cells were created in accordance with Example II and then screened against a PAD4 Arg⁶³⁹/His⁶⁴⁰ library.

The above data from Arg³⁷⁴/Arg⁶³⁹ library screening revealed that Arg³⁷⁴ may be involved in arginine binding, for example, by coordinating the carboxy terminus of the substrate. Thus, by taking an iterative approach, the method in this example left Arg³⁷⁴ intact and two other residues within 3 Å of the ligand binding site were mutated (i.e., for example, Arg⁶³⁹ and His⁶⁴⁰) and then screened to identify clones having improved arginine deiminase activity.

A PAD4 library was constructed by overlap extension PCR using the following oligonucleotides: Two complementary end primers were used according to the techniques described in Example II:

| | |
|---|---|
| i) | 5′-cttcacctaccacatcNNSNNSggggaggtgcactg-3′, |
| and | |
| ii) | 5′-CAGTGCACCTCCCCSNNSNNGATGTGGTAGGTGAAG-3′ |

These primers were used to introduce random codons into the positions corresponding to Arg⁶³⁹ and His⁶⁴⁰. The oligonucleotides generated from PCR using these primers were inserted into pGEX-6p1 vectors in accordance with the techniques described in Example II. The resulting vector library transformed DH5α E. coli cells and plated on LB-ampicillin plates which resulted in ˜12,000 individual clones. These plates were then scraped and mini-prepped to collect the library. The plasmid library was then transformed into Rosetta-2 E. coli cells and plated on LB plates containing 34 μg/ml chloramphenicol and 100 μg/ml ampicillin for subsequent screening.

After screening ˜1,000 clones, those clones exhibiting increased ADI activity were identified. Plasmid DNA was isolated from those respective E. coli cells, and sequenced to determine the mutations conferring the desired activity. Several variants displaying activity from the Arg³¹⁴/Arg⁶³⁹ library were obtained. See, Table 2.

TABLE 2
List of variants found from screen of PAD-R⁶³⁹/H⁶⁴⁰ library.

| WT | Arg⁶³⁹ | His⁶⁴⁰ | Times found |
|---|---|---|---|
| 1 | Arg | Gly | 3 |
| 2 | Arg | Asn | 1 |
| 3 | Lys | Asn | 1 |
| 4 | Lys | Val | 1 |
| 5 | His | Lys | 2 |
| 6 | Met | Arg | 1 |
| 7 | Lys | Arg | 1 |
| 8 | Arg | Lys | 1 |
| 9 | Val | Gly | 1 |
| 10 | Ile | Gly | 1 |
| 11 | Tyr | His | 1 |

### Example VI

### PAD4 Iterative Error Prone Library

This example presents one embodiment of a method to isolate ADI variants of PAD4 from libraries of random mutants.

Generally, random mutagenesis is carried out by means of error prone PCR. In one iterative approach, a PAD4 enzyme is mutagenized by error prone PCR, cloned into an appropriate vector, and the library is screened for ADI activity. Plasmids from clones displaying arginine degrading activity are pooled and subjected to further rounds of random mutagenesis etc.

Providing: i) the aforementioned end primers (Examples II and/or III); ii) Taq DNA polymerase and associated buffers (New England Biolabs, Beverly Mass.); iii) biased concentrations of dNTPs in the presence of Mg²⁺, Mn²⁺ and BSA (bovine serum albumin), the PAD4 gene is sufficiently amplified after 20-25 rounds of the PCR reaction. After cloning (as described above), ˜1000 clones are screened in accordance with Example I. Clones displaying ADI activity are sequenced to determine the nature of the mutation conferring activity. These clones are then tested for activity against L-arginine and benzoyl-L-arginine. Plasmids from clones displaying only ADI activity are pooled and used as the template for the next round of error prone mutagenesis.

Repeated rounds of mutagenesis increase the number of active clones, wherein the assay conditions are made more stringent by decreasing both the concentration of L-Arg and the reaction time, thereby ensuring selection of the most active clones. After several rounds of iterative error prone mutagenesis the identified most active clones are shuffled with wild-type sequence and re-screened. This allows recombination of the most advantageous mutations to ADI activity and edits out various extraneous mutations.

### Example V

### Expression and Purification of PAD4 and Variants

This example presents one embodiment of the expression and purification of PAD4 mutated enzymes.

Typically, PAD4 and variant proteins are expressed and purified as follows. An overnight culture of E. coli (i.e., for example, Rosetta-2 cells) harboring a pGEX-PAD4 variant plasmid of interest is used to inoculate TB medium (1 L) containing ampicillin (100 μg/ml) and chloramphenicol (34 μg/ml) and incubated in shake flasks (300 rpm) at 37° C. until the cell density reaches an OD₆₀₀ of ˜1. The culture is then cooled to 25° C., and IPTG (0.3 mM) is added to induce expression. After 4-12 h of continued incubation and expression at 25° C., cells are harvested by centrifugation and the cell pellets frozen at −20° C. Frozen cell pellets from 1 L of culture medium are resuspended in 300 mL of binding buffer [140 mM NaCl, 2.7 mM KCl, 10 mM Na₂HPO₄, and 1.8 mM KH₂PO₄ (pH 7.3)].

Cell suspensions are then lysed by sonication or by French pressure cell. Cell debris is removed by centrifugation at 23,500 g for 30 min. The resulting supernatant is diluted with ˜200 mL of binding buffer, loaded onto a 5-10 mL glutathione-Sepharose-4 fast flow affinity resin column (Amersham Biosciences), and washed with 10 column volumes of binding buffer. The fusion proteins are then eluted with reduced glutathione (10 mM) in Tris-HCl buffer (50 mM) and dithiothreitol (1 mM) at pH 8.0. Fractions containing active fusion proteins are pooled and dialyzed against three changes of 4 L of Tris-HCl (100 mM, pH 7.6) to remove excess glutathione. From 1 L of culture medium, this procedure yields 22 mg of purified GST-PAD4 fusion protein that is >90% homogeneous as assessed by SDS-PAGE (17).

### Example VI

### Determining Michaelis Kinetics and Substrate Specificity

This example illustrates one embodiment of characterizing mutagenized proteins.

Plasmids containing isolated PAD4 variants were re-transformed into Rosetta-2 cells (Novagen, Madison, Wis.) for large scale protein expression. The soluble fraction was then assayed using L-Arg or the peptidyl-arginine substrate analog benzoyl-L-arginine to determine both the Michaelis constant (K_{M}) and the substrate specificity of the mutant enzyme.

PAD4 variants were grown in 50 ml of TB media containing 34 μg/ml chloramphenicol and 100 μg/ml ampicillin at 37° C. until reaching an OD₆₀₀ of 0.8-1, whereupon they were cooled to 25° C. and induced with 0.3 mM IPTG for 3-4 hours. Cultures were collected by centrifugation, followed by re-suspension of the cell pellet in 10 ml of a 100 mM Tris buffer pH 7.6. After lysing by passing through a French pressure cell, the resulting material was centrifuged at 23,500×g for 30 min.

The cleared lysates were added to 96 well plates containing dilutions of L-arg or benzoyl-L-arginine (final concentration ˜10 mM-10 μM) in 100 mM Tris buffer containing 10 mM CaCl₂, and 5 mM DTT at pH 7.6. After reacting for 1 hr at 37° C., 100 μl/well of color developing reagent were added and the plate processed as described elsewhere. All reactions were done in at least triplicate. After measuring the absorbance at 530 nm, and subtracting the background contributions of supernatant and substrate, the resulting data was fit to the Michaelis-Menten equation. Several variants were found and their respective ability to hydrolyze either L-arginine or benzoyl-L-arginine was measured. See, Table 3.

TABLE 3
List of screened variants showing affinities to
L-arg or a peptidyl-arginine substrate analog; benzoyl-L-arg.

| PAD4 variants | | L-Arg | Benzoyl-L-Arg | |
|---|---|---|---|---|
| Pos 374 | Pos 639 | Km (μM) | Km (μM) | |
| Arg | Arg | NA | 400 | WT |
| Lys | His | A | NA | |
| Arg | Ala | 6000 | | |
| Glu | Gly | 1600 | NA | |
| Arg | Asn | NA | 3900 | |
| Arg | Glu | 800 | ND | |

NA = no activity,
A = active but non-saturating under assay conditions
WT = Wild Type

### REFERENCES

(1) Milner, J. A. (1985) Metabolic aberrations associated with arginine deficiency. J Nutr 115, 516-23.
(2) Muller, H. J., and Boos, J. (1998) Use of L-asparaginase in childhood ALL. Crit. Rev Oncol Hematol 28, 97-113.
(3) Avramis, V. I., and Panosyan, E. H. (2005) Pharmacokinetic/pharmacodynamic relationships of asparaginase formulations: the past, the present and recommendations for the future. Clin Pharmacokinet 44, 367-93.
(4) Wheatley, D. N., and Campbell, E. (2002) Arginine catabolism, liver extracts and cancer. Pathol Oncol Res 8, 18-25.
(5) Ensor, C. M., Holtsberg, F. W., Bomalaski, J. S., and Clark, M. A. (2002) Pegylated arginine deiminase (ADI-SS PEG20,000 mw) inhibits human melanomas and hepatocellular carcinomas in vitro and in vivo. Cancer Res 62, 5443-50.
(6) Sugimura, K., Ohno, T., Kusuyama, T., and Azuma, I. (1992) High sensitivity of human melanoma cell lines to the growth inhibitory activity of mycoplasmal arginine deiminase in vitro. Melanoma Res 2, 191-6.
(7) Yoon, C. Y., Shim, Y. J., Kim, E. H., Lee, J. H., Won, N. H., Kim, J. H., Park, I. S., Yoon, D. K., and Min, B. H. (2006) Renal cell carcinoma does not express argininosuccinate synthetase and is highly sensitive to arginine deprivation via arginine deiminase. Int J Cancer.
(8) Takaku, H., Takase, M., Abe, S., Hayashi, H., and Miyazaki, K. (1992) In vivo anti-tumor activity of arginine deiminase purified from Mycoplasma arginini. Int J Cancer 51, 244-9.
(9) Gong, H., Zolzer, F., von Recklinghausen, G., Havers, W., and Schweigerer, L. (2000) Arginine deiminase inhibits proliferation of human leukemia cells more potently than asparaginase by inducing cell cycle arrest and apoptosis. Leukemia 14, 826-9.
(10) van Rijn, J., van den Berg, J., Schipper, R. G., de Jong, S., Cuijpers, V., Verhofstad, A. A., and Teerlink, T. (2004) Induction of hyperammonia in irradiated hepatoma cells: a recapitulation and possible explanation of the phenomenon. Br J Cancer 91, 150-2.
(11) Holtsberg, F. W., Ensor, C. M., Steiner, M. R., Bomalaski, J. S., and Clark, M. A. (2002) Poly(ethylene glycol) (PEG) conjugated arginine deiminase: effects of PEG formulations on its pharmacological properties. J Control Release 80, 259-71.
(12) Ascierto, P. A., Scala, S., Castello, G., Daponte, A., Simeone, E., Ottaiano, A., Beneduce, G., De Rosa, V., Izzo, F., Melucci, M. T., Ensor, C. M., Prestayko, A. W., Holtsberg, F. W., Bomalaski, J. S., Clark, M. A., Savaraj, N., Feun, L. G., and Logan, T. F. (2005) Pegylated arginine deiminase treatment of patients with metastatic melanoma: results from phase I and II studies. J Clin Oncol 23, 7660-8.
(13) Tsao, H., Atkins, M. B., and Sober, A. J. (2004) Management of cutaneous melanoma. N Engl J Med 351, 998-1012.
(14) Izzo, F., Marra, P., Beneduce, G., Castello, G., Vallone, P., De Rosa, V., Cremona, F., Ensor, C. M., Holtsberg, F. W., Bomalaski, J. S., Clark, M. A., Ng, C., and Curley, S. A. (2004) Pegylated arginine deiminase treatment of patients with unresectable hepatocellular carcinoma: results from phase I/II studies. J Clin Oncol 22, 1815-22.
(15) Nakayama-Hamada, M., Suzuki, A., Kubota, K., Takazawa, T., Ohsaka, M., Kawaida, R., Ono, M., Kasuya, A., Furukawa, H., Yamada, R., and Yamamoto, K. (2005) Comparison of enzymatic properties between hPADI2 and hPADI4. Biochem Biophys Res Commun 327, 192-200.
(16) Vossenaar, E. R., Zendman, A. J., van Venrooij, W. J., and Pruijn, G. J. (2003) PAD, a growing family of citrullinating enzymes: genes, features and involvement in disease. Bioessays 25, 1106-18.
(17) Stone, E. M., Schaller, T. H., Bianchi, H., Person, M. D., and Fast, W. (2005) Inactivation of two diverse enzymes in the amidinotransferase superfamily by 2-chloroacetamidine: dimethylargininase and peptidylarginine deiminase. Biochemistry 44, 13744-52.
(18) Luo, Y., Arita, K., Bhatia, M., Knuckley, B., Lee, Y. H., Stallcup, M. R., Sato, M., and Thompson, P. R. (2006) Inhibitors and inactivators of protein arginine deiminase 4: functional and structural characterization. Biochemistry 45, 11727-36.
(19) Kearney, P. L., Bhatia, M., Jones, N. G., Yuan, L., Glascock, M. C., Catchings, K. L., Yamada, M., and Thompson, P. R. (2005) Kinetic characterization of protein arginine deiminase 4: a transcriptional corepressor implicated in the onset and progression of rheumatoid arthritis. Biochemistry 44, 10570-82.
(20) Knipp, M., and Vasak, M. (2000) A calorimetric 96-well microtiter plate assay for the determination of enzymatically formed citrulline. Anal Biochem 286, 257-64.

We claim:

1. A composition comprising a mutated human peptidyl arginine deiminase IV enzyme, wherein said enzyme comprises a high affinity free arginine binding site.

2. The composition of claim 1, wherein said mutated enzyme comprises at least two altered amino acid residues when compared to a wild type human peptidyl arginine deiminase IV enzyme.

3. The composition of claim 1, wherein said mutated enzyme comprises catalytic activity in the hydrolysis of arginine.

4. The composition of claim 2, wherein said altered amino acid residue comprises AA³⁷⁴.

5. The composition of claim 4, wherein said AA³⁷⁴ is selected from the group consisting of lysine, serine, and proline.

6. The composition of claim 2, wherein said altered amino acid comprises AA⁶³⁹.

7. The composition of claim 6, wherein said AA⁶³⁹ is selected from the group consisting of asparagine, lysine, serine, glutamic acid, histidine, methionine, valine, isoleucine, or tyrosine.

8. The composition of claim 2, wherein said altered amino acid comprises AA⁶⁴⁰.

9. The composition of claim 8, wherein said AA⁶⁴⁰ is selected from the group consisting of glycine, asparagine, valine, lysine, and arginine.

10. A composition comprising a human peptidyl arginine deiminase IV enzyme comprising at least two mutations, wherein said mutations are at amino acid positions selected from the group consisting of Arg³⁷⁴, Arg⁶³⁹, and His⁶⁴⁰.

11. The composition of claim 10, wherein said enzyme further comprises a high affinity free arginine binding site.

12. The composition of claim 10, wherein said enzyme comprises arginine deiminase activity.

13. The composition of claim 11, wherein said Arg³⁷⁴ mutation creates a first altered amino acid selected from the group consisting of lysine, serine, and proline.

14. The composition of claim 11, wherein said Arg⁶³⁹ mutation creates a second altered amino acid selected from the group consisting of asparagine, lysine, serine, glutamic acid, histidine, methionine, valine, isoleucine, and tyrosine.

15. The composition of claim 11, wherein said His⁶⁴⁰ mutation creates a third altered amino acid selected from the group consisting of glycine, asparagine, valine, lysine, and arginine.

16. A method, comprising:
a) providing a wild type nucleic acid sequence encoding a wild type human amino acid sequence, wherein said wild type amino acid sequence comprises a high catalytic activity for peptidyl arginine; and
b) mutagenizing the wild type nucleic acid sequence to create a mutated nucleic acid sequence, wherein said mutated nucleic acid sequence encodes a mutated human amino acid sequence, wherein said mutated amino acid sequence comprises high catalytic activity for L-Arg.

17. The method of claim 16, wherein said mutated human amino acid sequence comprises at least 95% of said wild type human amino acid sequence.

18. The method of claim 16, wherein said wild type human amino acid sequence comprises an peptidyl arginine deiminase IV enzyme.

19. The method of claim 16, wherein said mutated human amino acid sequence comprises a k_{cat} of 4-6 s⁻¹ for free arginine.

20. The method of claim 16, wherein said mutated human amino acid sequence comprises at least two altered amino acid residues.

21. A method, comprising:
a) providing:
i) a library of bacterial cells transfected by oligonucleotides encoding a mutated human peptidyl arginine deiminase IV enzyme; and
ii) an assay capable of detecting free arginine deiminase activity;
b) expressing said oligonucleotides from said bacterial cells, thereby producing said mutated enzymes; and
c) using said assay to identify said bacterial cells expressing said mutated enzymes, wherein said mutated enzymes metabolize free arginine.

22. The method of claim 21, wherein said bacterial cell comprise E. coli cells.

23. A method, comprising:
a) providing;
i) a human patient comprising a population of cancer cells, wherein said cancer cells are susceptible to an arginine deficiency;
ii) a mutated human peptidyl arginine deiminase IV enzyme, wherein said enzyme is capable of degrading free arginine; and
b) administering said enzyme to said patient under conditions that said population of cancer cells is reduced.

24. The method of claim 23, wherein said administering further creates said arginine deficiency.

25. The method of claim 23, wherein said enzyme is mutated at least two amino acid residues.

26. The method of claim 23, wherein said population of cancer cells comprise hepatic carcinoma cancer cells.

27. The method of claim 23, wherein said population of cancer cells comprise renal carcinoma cancer cells.
